(19) United States
(12) Patent Application Publication
(10) Pub. No.: US 2008/0255099 A1
(43) Pub. Date: Oct. 16, 2008

(54) Use of Diketodithiopiperazine Antibiotics for the Preparation of Antiangiogenic Pharmaceutical Compositions

(75) Inventors: Sergio De Munari, Milano (IT); Mario Grugni, Novate (IT); Ernesto Menta, Cernusco Sul Naviglio (IT); Mara Cassin, Milano (IT); Gennaro Colella, Bresso (IT)

Correspondence Address: SEED INTELLECTUAL PROPERTY LAW GROUP PLLC, 701 FIFTH AVE, SUITE 5400, SEATTLE, WA, 98104, US

(73) Assignee: Cell Therapeutics, Inc., Seattle, WA (US)

(21) Appl. No.: 11/793,860

(22) PCT Filed: Dec. 14, 2005
(86) PCT No.: PCT/EP05/13457

§ 371 Date: Jan. 25, 2008

(30) Foreign Application Priority Data
Dec. 23, 2004 (IT) M12004A002477

(51) Int. Cl. A61K 31/548; C12N 5/06

(52) U.S. Cl. 514/222.8; 435/375

(57) ABSTRACT

The invention relates to the use of diketodithiopiperazine antibiotics, in particular chaetocin and gliotoxin, for the preparation of pharmaceutical compositions for antitumor therapy.

The present invention relates to the use of diketodithiopiperazine antibiotics, in particular chaetocin and gliotoxin, for the preparation of medicaments with antiangiogenic activity.

### STATE OF THE ART

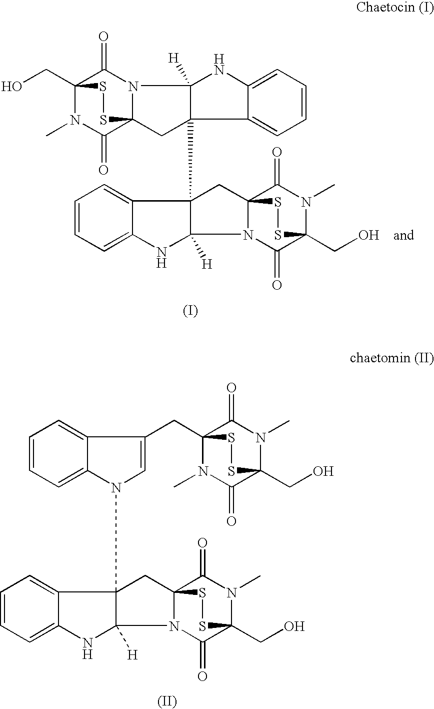

are representative examples of epipolythiodioxopiperazine antibiotics, which are secondary metabolites of moulds having anti-nicrobic and cytotoxic activity produced by fungi of the Chaetomium strain (C. Leigh, A. Taylor, Mycotoxins and other fungal metabolites related food problems, ed. J. V. Rodricks, p. 228, Am. Chem. Soc., Washington, D.C., 1976; G. W. Kirby, D. J. Robins, The Biosynthesis of Mycotoxins, ed. P. S. Stenyl, p. 301, Academic Press, New York, 1980. For the isolation of chaetocin from coltures of Chaetomium sp. strains, assigned to a C. thielavioideum, and from a Farrowia sp. strain, see also S. Udagawa et al., The production of chaetoglobosins, sterigmatocystin, O-methylsterigmatocystin, and chaetocin by Chaetomium spp. and related fungi, Can. J. Microbiol. 1979, 25(2):170-7 and S. Sekita, et al., Mycotoxin production by Chaetomium spp. and related fungi, Can. J. Microbiol. 1981, 27(8):766-72). The compounds of this class are characterised by the presence of a disulphide bond.

Besides chaetocin and chaetomin, further examples of epipolythiodioxopiperazines are gliotoxin (III)

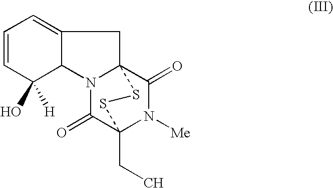

(P. Waring, J. Baever, Gliotoxin and related epipolithiodioxopiperazines, Gen Pharmacol. 27, 1311-1316, 1996), sporidesmine (Chem. Ber. 105(11): 3658-61, 1972), aranotine (N. Neuss et al., Aranotin and related metabolites. II. Isolation, Characterization and structures of two new metabolites, Tetrahedron Letters, 42, 4467-4471, 1968), verticillin (Chem. Ber. 105(11):3658-61, 1972;), melinacidin (F. Reusser, Mode of Action of Melinacidin, an Inhibitor of Nicotinic Acid Biosynthesis; J. Bacteriol. 96(4): 1285-1290, 1968) and oryzachlorin (also known as antibiotic A-30641 or aspirochlorine: K. Sakata et al., Structural revision of aspirochlorine (=antibiotic A30641), a novel epidithiopiperazine-2,5-dione produced by aspergillus SPP, Tetrahedron Letters, 28 (46), 5607-5610, 1987). Also a metabolite from Penicilium turbatum disclosed by K. Michel et al. in J. Antibiot. 27, 57 (1974) has a epipolythiodioxopiperazine structure.

Chaetocin's structure and absolute configuration have been disclosed by H. P. Weber (Helv. Chim. Acta, 53(5):1061-73, 1970; Acta Crystallogr. B28, 2945 (1972)). Cytotoxic activity of chaetocin and a dihydroxy derivative thereof, 11α,11α′-dihydroxy Chaetocin (Melinacidin IV) has been reported, the IC₅₀ being of about 0.03 μg/mL towards leukemic cells HeLa (T. Saito et al, Chetracin A, a new epipolithiodioxopiperazine having a tetrasulfide bridge from Chaetomium abuense and C. retardatum, Tetrahedron Letters, 26, (39), 4731-4734, 1985).

Vascular Endothelial cell Growth Factor (VEGF) plays a fundamental role in processes of physiological and physiopathological angiogenesis. Different mechanisms are involved in the regulation of the VEGF gene; among them tissue oxygen tension is highly relevant, as demonstrated by the reversibile increase in VEGF mRNA levels under in vivo and in vitro hypoxia conditions. The increased expression of VEGF mRNA is mainly mediated by the transcription hypoxia-inducible factor-1 (Hif-1), which binds to a recognition site in the promoter region of the VEGF gene.

A great number of experimental data show that Hif-1 is a global regulator of oxygen homeostasis and that an impaired Hif-1 activity promotes survival, proliferation, invasivity and metastatization of tumoral cells (G. L. Semenza, Nature Review Cancer, 3, 2003, 721-732). It has been therefore hypothesized that therapeutic strategies aimed at inhibiting Hif-1 activity can increase survival of cancer patients (Semenza G L. HIF-1 and tumor progression: pathophysiology and therapeutics, Trends Mol. Med. 2002 8:S62).

HIF-1 is a heterodimer consisting of Hif-1α and Hif-1β sub-units, which dimerize and bind DNA through the bHLH-PAS domain (Semenza G L et al. Dimerization, DNA binding, and transactivation properties of hypoxia-inducible factor 1, J. Biol. Chem. 1996 271:17771). The expression of the Hif-1α sub-unit is strictly regulated by tissutal oxygen (Semenza G L et al., Hypoxia-inducible factor 1 levels vary exponentially over a physiologically relevant range of O₂ tension, Am. J. Physiol. 1996 271:C1172), by processes of ubiquitination and proteosomal degradation mediated by the binding of the VHL protein to Hif-1α. This interaction occurs only when Hif-1α has been hydroxylated at the 402 and 564 proline residues. Oxygen is the limiting substrate for the prolyl-hydroxylase that modifies Hif-1α(Epstein A C et al. C. elegans EGL-9 and mammalian homologs define afamily of dioxygenases that regulate HIF by prolyl hydroxylation, Cell 2001 107:43). The expression of Hif-1α exponentially increases as O₂ concentration decreases and determines the global levels of Hif-1 activity.

The function of the Hif-1α transactivation domain is also subject to negative regulation controlled by oxygen partial pressure. The N-terminal transactivation domain is negatively regulated through the recruitment of hystone deacilase by VHL and the factor inhibiting Hif-1 (FIH-1), which binds to both VHL and Hif-1α (Semenza G L. et al. FIH-1: a novel protein that interacts with HIF-1alpha and VHL to mediate repression of HIF-1 transcriptional activity, Genes Dev. 2001 15:2675).

Hif-1 activation occurs through coactivators p300/CBP which physically interact with the activation of the Hif-1 domain to facilitate transcription of genes like VEGF (Arany Z. et al. An essential role for p300/cbp in the cellular-response to hypoxia, Proc. Nat. Acad. Sci. USA 1996 93; 12969). Both p300 and CBP are co-activators also for other transcription factors, such as Stat-3, NF-κB, p53.

The interaction of p300/CBP with Hif-1 is essential to transcription, and recent pubblications have proved the importance of the Hif-1/p300 interaction for tumor growth (Damert A. et al. Activator-protein-1 binding potentiates the hypoxia-inducible factor-1-mediated hypoxia induced transcriptional activation of vascular-endothelial growth-factor expression in c6 glioma cells, Biochem J. 1997 327:419). Hif-1α C-terminal trans-activation domain (C-TAD) binds to a p300 and CBP domain known as CH1. The binding of CBP and p300 to Hif-1α is negatively regulated through oxygen-dependent hydroxylation of the 803 asparagine in the C-terminal activation domain by FIH-1. Thus, hypoxia induces both stabilization to proteosome degradation and transcriptional activity of Hif-1.

The structural details of the interaction between Hif-1α TAD-C with the CH1 domain of p300 or CBP have been clarified (Eck M J. et al. Structural basis for recruitment of CBP/p300 by hypoxia-inducible factor-1alpha, Proc. Natl. Acad. Sci. USA, 2002 99:5367, Wright P E et al. Structural basis for Hif-1 alpha/CBP recognition in the cellular hypoxic response, Proc. Nat. Acad. Sci. USA, 2002 99:5271). Details of the interaction between p300/CBP and the CITED2 protein (also known as p35^{srj}), which is considered a negative regulator of HIF-1α activity, have also been published (Freedman, S. J. et al, Nature Structural Biology, 2003, 10(7), 504-12).

Hif-1 activation induces transcription of a number of genes involved in the production of angiogenic factors, glucose carriers, glycolytic enzymes, survival, migration and invasion factors, which are particularly important for tumor progression.

Aberrant expression of Hif-1α protein has been observed in more than 70% human tumors and their metastasis and has been associated to an increase in vascularization and tumor progression (Zhong, H. et al., Overexpression of hypoxia-inducible factor 1α in common human cancers and their metastases, Cancer Research, 1999, 59, 5830-5; Bos, R. et al., Levels of hypoxia-inducile-factors-1αduring breast carcinogenesis, J. Nat. Cancer Inst. 2001, 93, 309-14; Talks, K. I. et al., The expression and distribution of the hypoxia-inducible-factors HIF-1α and HIF-2 in normal human tissues). In clinical practice, aberrant expression of Hif-1α has been associated to therapy failure and mortality increase in a number of tumoral pathologies, such as non-small cells lung carcinoma (Giatromanolaki, A. et al., Relation of hypoxia inducible factor 1α and 2α in operable non-small cell lung cancer to angiogenic/molecular profile of tumors and survival, Br. J. Cancer 85, 881-890 (2001)), oro-pharyngeal squamous cell cancer (Aebersold, D. M. et al. Expression of hypoxia-inducible factor 1α: a novel predictive and prognostic parameter in the radiotherapy of oropharyngeal cancer, Cancer Res. 61, 2911-2916 (2001)), early stage cervical cancer (Birner, P. et al.

Overexpression of hypoxia-inducible factor 1α is a marker for an unfavorable prognosis in early-stage invasive cervical cancer, Cancer Res. 60, 4693-4696 (2000)), head-and-neck cancer (Koukourakis, M. I. et al., Hypoxia-inducible factor (Hif1A and Hif2A), angiogenesis, and chemoradiotherapy outcome of squamous cell head-and-neck cancer, Int. J. Radiat. Oncol. Biol. Phys. 53, 1192-1202 (2002)), mutated-p53 ovary cancer (Birner, P. et al., Expression of hypoxia-inducible factor 1α in epithelial ovarian tumors: its impact on prognosis and on response to chemotherapy, Clin. Cancer Res. 7, 1661-1668 (2001)), oligodendrioglioma (Birner, P. et al., Expression of hypoxia-inducible factor-1α in oligodendrogliomas: its impact on prognosis and on neoangiogenesis, Cancer 92, 165-171 (2001)) and BCL-2-positive esophageal cancer (Koukourakis, M. I. et al., Hypoxia inducible factor (HIF-1αand HIF-2α) expression in early esophageal cancer and response to photodynamic therapy and radiotherapy, Cancer Res. 61, 1830-1832 (2001)).

Different approaches for inhibiting Hif-1 activity have been described in the literature. Some of them suggested the use of antisense oligonucleotides for Hif-1α or of negative dominant HIF-1α forms.

Among the pharmacological approaches, Hif-1α activity inhibitors acting through indirect mechanisms have been described, such as: PI3K-mTOR inhibitors (Zundel, W. et al. Loss of PTEN facilitates HIF-1-mediated gene expression, Genes Dev. 14, 391-396 (2000); Hudson, C. C. et al. Regulation of hypoxia-inducible factor 1-alpha expression and function by the mammalian target of rapamycin, Mol. Cell. Biol. 22, 7004-7014 (2002)) and MEKK inhibitors (Sodhi, A. et al. The Kaposi's sarcoma-associated herpes virus G protein-coupled receptor up-regulates vascular endothelial growth factor expression and secretion through mitogen-activated protein kinase and p38 pathways acting on hypoxia-inducible factor 1α, Cancer Res. 60, 4873-4880 (2000)) which act on the transduction of signals that control Hif-1α activity; inhibitors of HSP90 chaperone protein (Mabjeesh, N. J. et al. Geldanamycin induces degradation of hypoxia-inducible factor 1α protein via the proteosome pathway in prostate cancer cells, Cancer Res. 62, 2478-2482 (2002)); inhibitors of thioredoxin-reductase, which modify the cellular redox state (Welsh, S. J. et al. The thioredoxin redox inhibitors 1-methylpropyl 2-imidazolyl disulfide and pleurotin inhibit hypoxia-induced factor 1α and vascular endothelial growth factor formation, Mol. Cancer. Ther. 2, 235-243 (2003)); molecules which destabilize microtubules, such as 2-methoxyestradiol (Mabjeesh, N. J. et al. 2ME2 inhibits tumor growth and angiogenesis by disrupting microtubules and dysregulating Hif, Cancer Cell 3, 363-375 (2003)) and epothilones (Escuin, D. et al., Epothilone B inhibits Hif-1α downstream of its microtubule stabilizing effects, Proceedings of the 95^{th} Annual Meeting of the American Association for Cancer Research, Abs. 5427).

Recently, inhibition of both constitutive and hypoxia-induced Hif-1α levels by PX-478 (Melphalan N-oxide) in human tumors transplanted from nude mice has been reported. The compound shows marked antitumoral activity. However, the mechanism of action of this compound has not yet been fully clarified (S Welsh et al, Antitumor activity and pharmacodynamic properties of PX-478, an inhibitor of hypoxia-inducible factor 1α, Mol Cancer Ther. 3:233-244, (2004)).

Finally, it has recently been reported that chaetomin-a metabolite of Chaetomium sp fungi with dithiodiketopiperazine structure-interferes with the binding of Hif-1α to p300. The compound acts altering the structure of the CH1 domain of p300, thus preventing its interaction with Hif-1α. Administration of chaetomin to tumor-bearing mice inhibits hypoxia-induced transcription in the tumor and tumor growth (A. L. Kung et al., Cancer Cell, 6, 33-43, 2004).

Gliotoxin and chaetocin are commercially available from Sigma Aldrich and can be obtained according to the methods described in the above-mentioned publications. The total synthesis of gliotoxin is reported by T. Fukuyama, S, Nakatsuka e Y. Kishi in Total synthesis of gliotoxin, dehydrogliotoxin and hyalodendrin, Tetrahedron, 37(11), 2045-2078, 1981.

### DISCLOSURE OF THE INVENTION

It has now been found that antibiotics with a diketodithiopiperazine structure, in particular chaetocin and gliotoxin, are able to inhibit the binding of Hif-1α with p300 and to prevent VEGF production in cells maintained under hypoxia conditions.

Thus, in a first embodiment the invention relates to the use of diketodithiopiperazine antibiotics selected from chaetocin and gliotoxin for the preparation of medicaments for the treatment of pathologies wherein inhibition of the binding of Hif-1α with p300, in particular for the preparation of antiangiogenic medicaments.

Object of the invention are therefore chaetocin and gliotoxin as anti-angiogenic, anti-proliferative and anti-metastatic agents.

In a further embodiment, the invention relates to pharmaceutical compositions comprising diketodithiopiperazine antibiotics selected from chaetocin and gliotoxin active ingredients, in admixture with suitable carriers and excipients.

The invention further relates to a method for inhibiting VEGF production in a cell, which method comprises contacting the cell with an effective amount of chaetocin or gliotoxin.

### DETAILED DESCRIPTION OF THE INVENTION

Diketodithiopiperazine antibiotics, in particular chaetocin and gliotoxin, are able to inhibit the interaction between Hif-1α and p300, as it has been possibile to demonstrate with a fluorescency assay adapted from Freedman S J et al., Nature Structural Biology 2003, 10 (7), 504-512.

Chaetocin and gliotoxin are therefore useful for the control of angiogenesis and tumor growth.

Pharmaceutical compositions of these compounds can be conveniently used for the treatment of a number of pathologies wherein angiogenesis is involved as pathogenesis factor, for example different forms of solid tumors, diabetis retinopathy, rheumatoid arthritis, psoriasis, emangioma, scleroderma, neovascular glaucoma.

Solid tumors that are particularly sensitive to compounds able to inhibit the binding of Hif-1α with the CH1 domain of p300 comprise lung carcinoma, mammarian carcinoma, prostate carcinoma, neuroblastoma, glioblastoma multiforme, melanoma, central nervous system tumors, oro-faryngeal squamous cell cancer, cervix, ovary, esophageal, kidney, colon, head-and-neck tumor and oligodendroglioma.

For the envisaged therapeutical uses, said ketodithiopiperazine antibiotics will be administered through the oral, parenteral, transdermal, rectal, topical or equivalent administration route, in dosages that will be determined by the experts in the field according to the pharmaco-toxicology and pharmacokinetic properties of the selected compound and according to the pathology, its severity and progression stage and to the patient's weight, sex and age.

However, the dosages will be typically comprised between 0.1 and 100 mg/Kg/die with respect to the weight of the patient.

Chaetocin and/or gliotoxin will optionally be administered in combination with other chemotherapeutic agents, for instance in chemotherapy protocols with potentially synergistic drugs having different mechanism of action.

Examples of compositions of the invention comprise capsules, tablets, injectable or oral solutions or suspensions, suppositories, controlled-released forms and the like. Said compositions can be prepared by means of conventional techniques and excipients, for example those disclosed in Remington's Pharmaceutical Sciences Handbook, XVII ed. Mack Pub., N.Y., U.S.A.

The invention is illustrated in greater detail in the following examples.

### Example 1

### Inhibition of Biot-Hif-1α⁷⁸⁶⁻⁸²⁶/GST-p300^{323/423}

Chaetocin's ability to prevent interaction between Hif-1α and p300 has been evaluated using the fluorescency assay (DELFIA™) method disclosed by Freedman S J at al., Nature Structural Biology 2003, 10 (7), 504-512, suitably modified.

The human biotinylated Hif-1α fragment corresponding to C-terminal aminoacids 786-826 (Biotinylated Hif-1α⁷⁸⁶⁻⁸²⁶) was obtained by AnaSpec Inc (San José, Calif., USA) and used without further purifications.

A construct expressing the GST-p300³²³⁻⁴²³ fragment was transformed in the BL21 (DE3) strain of E. coli. Such construct was obtained by cloning in the expression vector pGEX-4T-1 (Amersham n. 27-45-80-01) the DNA sequence which encodes for the p300 region comprised between the 323-423 aminoacids; the DNA sequence was obtained through PCR (Polymerase Chain Reaction). The expression of the protein was induced with 1 mM isopropyl-1-thio-β-D-galactopiranoside (IPTG). The bacteria were lysed through sonication in the presence of a suitable buffer (50 mM Tris.HCl pH 8.00, 100 mM NaCl, 0.1 mM ZnSO4, 1 mM DTT, 0.1 mg/ml lysozime and a tablet of Roche protease inhibitor) and GST fusion protein contained in the soluble fraction was purified on a Glutathione-Sepharose 4B resin (Amersham Biosciences; no. 27-4574-01). The protein final concentration was determined according to Bradford with the Biorad assay (Bradford M., Anal. Biochem., 72, 248, (1976)). Samples purity was evaluated through SDS-PAGE. The samples were stored at −80° C. in glycerol 50%.

The assay was carried out as follows, using 96-wells NUNC Maxisorp plates. C96 NUNC Maxisorp plates (Nunc, product No. 446612) were incubated overnight with streptavidin (Sigma; product No. S 4762) at a final concentration of 1 μg/ml in PBS buffer (Phosphate Buffered Saline 10 mM sodium phosphate, 150 mM sodium chloride pH 7.4). Each well was then washed three times with 300 μl of TBST buffer (50 mM Tris-HCl pH 8.0, 150 mM NaCl, 0.05% (v/v) Tween 20). Each well was then added with 100 μl of a 10 nM solution of biotinylated Hif-1α⁷⁸⁶⁻⁸²⁶ in TBSB (50 mM Tris-HCl pH 8.0, 150 mM NaCl, 5% (w/v) BSA (Sigma, product No. A 2153)) and incubated 1 h at 25° C. In the last row of each plate only TBSB buffer was added. Each well was then washed three times with 300 μl of TBST buffer. The plate so prepared was used for the assay.

Separately, a plate (daughter plate) containing in each well 10 μl of a 10 μM solution of each test compound in DMSO was prepared. This plate was added with 100 μl of a 111 pM solution of GST-p300³²³⁻⁴²³ diluted in incubation buffer (TBSB added with 0.1% (v/v) Tween 20, 0.5 mM DTT, 10 μM ZnCl₂), mixing the solutions. 100 μL of the mixture contained in the daughter plate were immediately transferred in the assay plate.

Each daughter plate was prepared with chaetocin at a concentration of 10 μM, safe for the two last well rows, wherein each well was added with 10 μL of DMSO. These two rows represented the positive (row 11, +Hif-1) and negative (row 12, -Hif-1) control.

After incubation for 1 h at 25° C., each well was washed three times with 300 μL TBST buffer (50 mM Tris-HCl pH 8.0, 150 mM NaCl, 0.05% (v/v) Tween 20). Each well was then added with 60.8 ng of an Europium-labeled anti-GST antibody (DELFIA Eu-N1 labeled; Perkin Elmer; product no. AD 0251) dissolved in 100 μL TBSB buffer containing 10 μM ZnCl₂. After incubation for 1 h at room temperature, each well was washed three times with 300 μL TBST buffer, then 100 μl of signal-amplifying solution (Enhancement Solution, Perkin Elmer prodotto No. 1244-105) was added.

The plates were then read with a FUSION alpha-FP-HT (Perkin Elmer) reader in fluorescence mode for time resolution.

Chaetocin activity was calculated as follows. The fluorescence mean value of negative controls in row 12 of the test plate was subtracted to the fluorescence value of all the other wells. The resulting fluorescence value for each well was then divided by the mean fluorescence value of the positive controls in row 11 (which represented the maximum signal value, 100%), and expressed as percent value. The inhibition value was the difference between 100 and the signal percentage calculated for each well.

Using daughter plates where the compounds were present at ten different concentrations comprised between 90 μM and 0.178 μM in each row, a dose-response curve could be calculated from which the IC₅₀ could be derived (concentration of the compound necessary to cause 50% inhibition of the signal). Rows 11 and 12 containing the vehicle only represented the controls.

In this test, chaetocin showed inhibition of the interaction between Biot-Hif-1α⁷⁸⁶⁻⁸²⁶ and GST-p300^{323/423} with an IC₅₀ of 12.5 μM.

### Example 2

### Inhibition of VEGF Production

The compounds of the invention were evaluated using a cellular test on genetically modified human epatocarcinoma Hep3B cells (Hep3B-VEGFLuciferase) in order to stably express a vector wherein luciferase Open Reading Frame is placed under the control of the rat VEGF gene promoter.

HIF-1 induction with deferoxamine (which induces hypoxia) induces luciferase trascription through activation of the VEGF promoter, which in turn leads to an increase of luciferase activity which can be measured with a commercially available kit. The compounds interfering with the HIF-1α/p300 complex cause inhibition of HIF-dependent luciferase activation, resulting in the reduction of luciferase activity. Therefore, this assay allows to evaluate the activity of the compounds towards the VEGF promoter, which is essential to VEGF production and subsequent tumor angiogenesis.

The Hep-3B-VEGF Luciferase line was obtained according to the following procedure.

Human epatocarcinoma Hep3B Cells (ATCC reference No. HB-8064) were seeded onto 6-well plates at a concentration of 2.5×10⁵ cells/well in 2 mL DMEM/10% FCS and the day after were transfected with Fugene 6 (Roche Biochemicals®). The transfection mixture in each well contained 6 μl of the transfection reaction Fugene 6, 1 μg of the reporter plasmid pxp2-VEGF-luciferase (rat VEGF promoter, NCBI GenBank accession No. U22373, Levy et al., J. Biol. Chem. 270 (22), 13333-13340, 1995), and 10 ng of pcDNA 3.1(+) plasmid (INVITROGEN) which makes cells resistant to neomycin. Transfection was carried out according to the manufacturer's instructions.

The suitable cell population (phenotypically resistant to neomycin) was selected through a cloning approach based on the “limit dilution” procedure (Sambrook J., Fritsch E. F. and Maniatis T. (1989) Molecular Cloning, A. Laboratory Manual; Cold Spring Harbor Laboratori). The following test of Luciferase expression/activity “Luciferase assay” and test for the quantification of VEGF secreted in the supernatant “Secreted VEGF ELISA test”) are carried out with stably transfected selected cells.

The following experimental protocol was used.

Day 1. Hep-3B-VEGF Luciferase cells were seeded onto “blank” 96-well plates (Greiner) at a density of 1×10⁴ cells/well/125 μl of medium, then allowed to adhere overnight in a thermostat (37° C./5% CO₂).

Day 2. 75 μl of “3.2× working solutions” of compound (previously prepared in medium so that DMSO concentration amounted to 1.6% v/v) was added to the cells (partial volume/well=200 μl, partial concentration the compound=1.2×, partial concentration of DMSO=0.6%). After 1 h incubation in thermostat, hypoxia was induced chemically by addition of 40 μl/well of a 6× (600 μM) stock solution of deferoxamine (final volume/well=240 μl, final concentration of the compound=1×, final concentration of DMSO=0.5%, final concentration of deferoxamine=1×≈100 μM). The plates were then placed in a thermostat for further 18-20 h.

Day 3. The “luciferase assay” and the “secreted VEGF ELISA test” were carried out as described in the following.

Secreted VEGF ELISA Test

Quantification of secreted VEGF was carried out using the “DuoSet Elisa Development System human VEGF” kit (R&D Systems).

100 μl/well of supernatant from the “blank” 96-well plates seeded with the cells of the Hep3B/VEGF Luciferase clone were transferred into transparent 96-well plates (Maxisorp) and assayed according to the instructions of the kit manufacturer.

In the ELISA test for inhibition of secreted VEGF chaetocin and gliotoxin showed IC₅₀ of 0.1 μM and 0.2 μM respectively.

Luciferase Assay

Quantification of expression of Luciferase reporter gene was carried out with Bright Glo Reagent (Promega). After discarding the supernatant and washing once with PBS, 40 μl/well of Bright Glo Reagent were added to “blank” 96-well plates, i.e. plates without human hepatocarcinoma Hep3B/VEGF-Luciferase cells. The reporter gene expression levels were determined reading the plates with a luminometer.

In the luciferase assay for the inhibition of the VEGF promoter chaetocin and gliotoxin showed an IC₅₀ (concentration of the compound that causes 50% inhibition of luciferase signal) of 0.04 μM and 0.05 μM respectively.

1.-3. (canceled)

4. A method for inhibiting vascular endothelial cell growth factor (VEGF) production in a cell, comprising contacting said cell with chaetocin in an amount effective to inhibit VEGF production.

5. A method for the prevention or treatment of angiogenesis or a tumor in an individual in need thereof, comprising administering chaetocin to the individual in an amount effective for prevention or treatment.

6. The method according to claim 5 wherein the tumor is a solid tumor.

7. The method according to claim 5 wherein the tumor is selected from lung carcinoma, mammary carcinoma, prostate carcinoma, neuroblastoma, glioblastoma multiforme, melanoma, central nervous system cancer, oro-pharyngeal squamous cell cancer, cervical, ovary, esophageal, kidney, colon, head-and-neck cancer and oligodendroglioma.

8. The method according to claim 5 wherein the chaetocin is together with a pharmaceutically acceptable vehicle or excipient.

9. The method according to any one of claims 5 to 8 wherein the individual is administered an other chemotherapeutic agent.
